SEQUENCE LISTING (12 sequences)

SEQ ID NO: 1 - DNA, 18 bp, Artificial sequence
  Primer
cggtggacaaattgtcac

SEQ ID NO: 2 - DNA, 22 bp, Artificial sequence
  Primer
cttctctggatttaacacactt

SEQ ID NO: 3 - DNA, 51 bp, Artificial sequence
  Primer
tcagcacacaaagccaaaaatttatttttctgtgcaaaggaaattaaggag

SEQ ID NO: 4 - DNA, 49 bp, Artificial sequence
  Primer
tattggtggagctaaacttaaagccttttctgtacaatccctttgagtg

SEQ ID NO: 5 - DNA, 28 bp, Artificial sequence
  Primer
ttacaagcttaaagaatgtctgaacact

SEQ ID NO: 6 - DNA, 27 bp, Artificial sequence
  Primer
ttgaatttaggtgaaacatttgtcacg

SEQ ID NO: 7 - DNA, 18 bp, Artificial sequence
  Primer
aacacaagctttcggcag

SEQ ID NO: 8 - DNA, 22 bp, Artificial sequence
  Primer
gaaatttggatctttgtcatcc

SEQ ID NO: 9 - DNA, 41 bp, Artificial sequence
  Primer
tgcggccaatgtttgtaatcagccaaggaaattttggggac

SEQ ID NO: 10 - DNA, 39 bp, Artificial sequence
  Primer
cgcattggcatggaagtcactttgatggcacctgtgtag

SEQ ID NO: 11 - DNA, 19 bp, Artificial sequence
  Primer
ttccttgtctgattagttc

SEQ ID NO: 12 - DNA, 18 bp, Artificial sequence
  Primer
accttcgggaacgtggtt

(19) United States
(12) Patent Application Publication
(10) Pub. No.: US 2024/0182987 A1
(43) Pub. Date: Jun. 6, 2024

(54) COMPOSITIONS AND METHODS FOR RAPID COVID-19 DETECTION

(71) Applicant: DUKE UNIVERSITY, Durham, NC (US)

(72) Inventors: Xin Song, Durham, NC (US); John Reif, Durham, NC (US)

(21) Appl. No.: 18/551,547

(22) PCT Filed: Mar. 21, 2022
(86) PCT No.: PCT/US22/21138

§ 371 Date: Sep. 20, 2023

Related U.S. Application Data
(60) Provisional application No. 63/164,098, filed on Mar. 22, 2021.

(51) Int. Cl. C12Q 1/70; C12Q 1/6844

(52) U.S. Cl. CPC C12Q 1/70; C12Q 1/6846

(57) ABSTRACT

The present disclosure provides compositions and methods related to the detection of pathogenic organisms. In particular, the present disclosure provides compositions and methods related to the detection and/or quantification of viral RNA in a sample from a subject that has, or is suspected of having, a SARS-COV-2 infection. Using rapid reverse-transcription loop-mediated isothermal amplification (RT-LAMP), the compositions and methods of the present disclosure provide a portable, inexpensive, rapid, and accurate assay platform for detecting and/or quantifying the presence of a pathogenic organism (e.g., SARS-COV-2) in a patient sample.

### CROSS REFERENCE TO RELATED APPLICATION

This application claims priority to and the benefit of U.S. Provisional Patent Application Ser. No. 63/164,098 filed Mar. 22, 2021, which is incorporated herein by reference in its entirety and for all purposes.

### GOVERNMENT FUNDING

This invention was made with Government support under Federal Grant nos. CCF 1617791 and CCF 1813805 awarded by the National Science Foundation. The Federal Government has certain rights to this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 2,382 Byte ASCII (Text) file named “40653-601_SEQUENCE_LISTING_ST25,” created on Mar. 21, 2022.

### FIELD

The present disclosure provides compositions and methods related to the detection of pathogenic organisms. In particular, the present disclosure provides compositions and methods related to the detection and/or quantification of viral RNA in a sample from a subject that has, or is suspected of having, a SARS-CoV-2 infection. Using rapid reverse-transcription loop-mediated isothermal amplification (RT-LAMP), the compositions and methods of the present disclosure provide a portable, inexpensive, rapid, and accurate assay platform for detecting and/or quantifying the presence of a pathogenic organism (e.g., SARS-CoV-2) in a patient sample.

### BACKGROUND

The COVID-19 pandemic has cost millions of lives and presented unprecedented economic, social, and structural challenges across the world. Whilst vaccination rollouts and improved access to testing have helped to control the pandemic, transmissions and infections (including post-vaccine breakthrough cases) due to the new variants of SARS-CoV-2 continue to pose a burden on global public health and economics. Effective SARS-CoV-2 surveillance requires frequent testing with rapid results to quickly identify infected individuals to break transmission chains. However, centralized testing models such as drive-through tests may increase the risk of exposure to health care workers and also rely on costly facilities, trained personnel, and sophisticated lab equipment (typically RT-qPCR) that in many cases still fail to deliver a timely test result. These delays can be detrimental to effective surveillance and disease control efforts due to the risk of presymptomatic/asymptomatic transmission of SARS-CoV-2. While testing labs are widely available in many high-income countries, most low- and middle-income countries lack sufficient facilities and trained personnel for wide-spread application of sophisticated SARS-CoV-2 detection technologies. Rapid antigen tests are easy to use and less expensive, however, while they are effective at screening symptomatic patients with high viral loads⁶, the overall higher rates of false positives and false negatives (compared to nucleic acid tests) make rapid antigen tests less suitable as front-line diagnostic. To quickly identify emerging SARS-CoV-2 transmission hotspots and curb the spread of virus from all potential transmission routes (presymptomatic, symptomatic, asymptomatic), a robust decentralized testing model would require the development of affordable nucleic acid home tests that are reliable, simple to use, and inexpensive to manufacture and distribute to large populations.

Since the beginning of the pandemic, researchers have sought to develop rapid molecular assays to overcome the practical limitations of standard RT-qPCR testing. Among several candidate nucleic acids amplification protocols, RT-LAMP is a simple method that achieves rapid exponential amplification of RNA using a set of six primers to recognize eight distinct regions on the target RNA sequence, enabling highly specific and sensitive detection of target RNA without stringent requirement on sample purity. This eliminates the need for the sophisticated RNA isolation and purification processes that have been a major bottleneck of current SARS-CoV-2 testing workflows. Further, the compatibility with simple pH-based colorimetric readout allows easy interpretation of the test result by visual inspection, making RT-LAMP suitable for inexpensive point-of-care applications. To date, many RT-LAMP assays have been proposed for SARS-CoV-2 detection including several that have obtained FDA Emergency Use Authorization (EUA). However, most of these tests still cannot meet the need for frequent at-home testing due to either un-optimized performance or the prohibitive cost per test.

### SUMMARY

Embodiments of the present disclosure include a composition for performing a reverse transcription loop-mediated isothermal amplification (RT-LAMP) reaction. In accordance with these embodiments, the composition includes a reaction buffer comprising a DNA polymerase and a reverse transcriptase; a chaotropic agent; and at least one excipient. In some embodiments, the composition is lyophilized to form an RT-LAMP reaction mixture.

In some embodiments, the chaotropic agent is selected from the group consisting of n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea. In some embodiments, the chaotropic agent is guanidine hydrochloride. In some embodiments, the guanidine hydrochloride is present at a concentration prior to lyophilization sufficient to produce a reconstituted concentration ranging from about 20 mM to about 80 mM.

In some embodiments, the at least one excipient is selected from the group consisting of sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide. In some embodiments, the excipient is trehalose. In some embodiments, the trehalose is present at a concentration prior to lyophilization sufficient to produce a reconstituted concentration ranging from about 5% w/v to about 20% w/v.

In some embodiments, the composition further comprises a visible pH indicator selected from the group consisting of cresol red, phenol red, neutral red, and m-cresol purple.

In some embodiments, the composition further comprises a LAMP primer mix comprising F3 primers, B3 primers, FIP primers, BIP primers, LoopF primers, and LoopB primers.

In some embodiments, the composition further comprises dNTPs, tris hydrochloride, ammonium sulfate, potassium chloride, magnesium sulfate, betaine, and tween 20.

In some embodiments, the RT-LAMP reaction mixture is mixed with a biological sample from a subject. In some embodiments, the biological sample is obtained from the subject's mouth and/or nasal cavity.

In some embodiments, the LAMP primer mix comprises primers specific for cDNA sequences corresponding to RNA from a pathogenic organism. In some embodiments, the pathogenic organism is an RNA virus. In some embodiments, the pathogenic organism is SARS-CoV-2.

In some embodiments, the primers comprise the following sequences: F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6.

Embodiments of the present disclosure also include a method for detecting a pathogenic organism in a biological sample from a subject. In accordance with these embodiments, the method includes: (a) combining in a reaction vessel a biological sample from a subject and the RT-LAMP reaction mixture described herein; (b) incubating the reaction vessel for at least 20 mins at a temperature of at least 60° C.; and (c) performing a visual inspection of the reaction vessel to determine if the biological sample is positive for the presence of a pathogenic organism.

In some embodiments, the method further comprises incubating the biological sample for at least 5 mins at a temperature of at least 90° C. prior to step (a).

In some embodiments of the method, the reaction vessel is insulated and configured to contain a liquid at a substantially constant temperature. In some embodiments of the method, the reaction vessel comprises a device for measuring the temperature of the liquid.

In some embodiments of the method, the RT-LAMP reaction mixture further comprises a visible pH indicator selected from the group consisting of cresol red, phenol red, neutral red, and m-cresol purple.

In some embodiments of the method, the RT-LAMP reaction mixture further comprises dNTPs, tris hydrochloride, ammonium sulfate, potassium chloride, magnesium sulfate, betaine, and tween 20.

In some embodiments of the method, the RT-LAMP reaction mixture further comprises a LAMP primer mix comprising F3 primers, B3 primers, FIP primers, BIP primers, LoopF primers, and LoopB primers. In some embodiments of the method, the LAMP primer mix comprises primers specific for cDNA sequences corresponding to RNA from a pathogenic organism.

In some embodiments of the method, the pathogenic organism is an RNA virus. In some embodiments, the pathogenic organism is SARS-CoV-2.

In some embodiments of the method, the biological sample is obtained from the subject's mouth and/or nasal cavity.

Embodiments of the present disclosure also include a method for generating a reverse transcription loop-mediated isothermal amplification (RT-LAMP) reaction mixture. In accordance with these embodiments, the method includes combining a reaction buffer comprising a DNA polymerase and a reverse transcriptase with a chaotropic agent and at least one excipient into a container; and subjecting the container to a lyophilization process to form an RT-LAMP reaction mixture.

In some embodiments of the method, the chaotropic agent is selected from the group consisting of n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea. In some embodiments of the method, the chaotropic agent is guanidine hydrochloride. In some embodiments of the method, the guanidine hydrochloride is present at a concentration prior to lyophilization sufficient to produce a reconstituted concentration ranging from about 20 mM to about 80 mM.

In some embodiments of the method, the at least one excipient is selected from the group consisting of sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide. In some embodiments of the method, the excipient is trehalose. In some embodiments of the method, the trehalose is present at a concentration prior to lyophilization sufficient to produce a reconstituted concentration ranging from about 5% w/v to about 20% w/v.

In some embodiments of the method, the reaction mixture further comprises a visible pH indicator selected from the group consisting of cresol red, phenol red, neutral red, and m-cresol purple.

In some embodiments of the method, the reaction mixture further comprises a LAMP primer mix comprising F3 primers, B3 primers, FIP primers, BIP primers, LoopF primers, and LoopB primers.

In some embodiments of the method, the reaction mixture further comprises dNTPs, tris hydrochloride, ammonium sulfate, potassium chloride, magnesium sulfate, betaine, tween 20.

In some embodiments of the method, the LAMP primer mix comprises primers specific for cDNA sequences corresponding to RNA from a pathogenic organism.

In some embodiments of the method, the pathogenic organism is an RNA virus. In some embodiments of the method, the pathogenic organism is SARS-CoV-2.

In some embodiments of the method, the primers comprise the following sequences: F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6.

### BRIEF DESCRIPTION OF THE DRAWINGS

FIG. 1: Schematic illustration of COVID-19 home test from sample collection to test result readout, according to one embodiment of the present disclosure.

FIG. 2: Comparison and optimization of different RT-LAMP formulations by thermal gradient test. All lyophilized (lyo) samples were stored at room temperature for 1 day before the RT-LAMP experiment. Fresh samples in solution (sol) were prepared at the time of the RT-LAMP experiment. For each set, the optimal incubation temperature was determined as the temperature that achieved both fast reaction (i.e., short time to true positives) and minimal false positives (i.e., long separation between true positives and false positives). False positives are marked by white asterisks. RT-LAMP reactions with optimal temperatures are shown in green boxes.

FIG. 3: Comparison and optimization of different RT-LAMP formulations based on a different primer set. All lyo samples were stored at room temperature for 1 day before the RT-LAMP experiment. Fresh samples were prepared at the time of the RT-LAMP experiment. Green boxes indicate tolerable incubation temperatures with reliable results. False positives are marked by white asterisks. The addition of 3M trehalose drastically reduced the false positives but slightly delayed the time to true positives. The lyophilized RT-LAMP reactions enabled a wider compatible range of incubation temperatures. Overall, the (3M trehalose+1M GuHCl) lyo set was optimal in terms of short time to true positives, wide range of tolerable temperatures, and low false positive rate.

FIGS. 4A-4B: Optimization of one-pot lyophilization for extended room temperature storage. (FIG. 4A) Physical appearance of the RT-LAMP reagents lyophilized with dextran added at different concentrations. (FIG. 4B) RT-LAMP tests conducted after storing the lyophilized test kit for 3 days at room temperature. Green boxes indicate RT-LAMP reactions at optimal temperatures with no false positives by 60 minutes. Blue boxes indicate RT-LAMP reactions that tolerate a wide range of temperatures for incubation under 50 minutes. False positives are marked by white asterisks.

FIG. 5: Comparison of two lyophilization formulations (with and without dextran) for extended room-temperature storage. Lyophilized test kits were stored at room temperature for 10 days and then reconstituted to run RT-LAMP. True negatives and true positives are labeled by (−) and (+), respectively. False positives are marked by white asterisks.

FIG. 6: Lyophilized test kit performance after 30-day storage in the fridge (4° C.) Analytical validation of test sensitivity and specificity for the optimal (3M+GuHCl) lyo formulation. True negatives and true positives are labeled by (−) and (+), respectively.

FIG. 7: Initial testing of NEB primer set by real-time LAMP. Fluorescence curves shown for single dilution series including two no template controls. Reactions done in solution. IDT gBlocks™ gene fragment was used as a proxy target template because SARS-CoV-2 control RNA was not commercially available at the time of this experiment. Copy number indicates total copies of target template per reaction.

FIG. 8: Initial testing of Shenyang primer set by real-time LAMP. Fluorescence curves shown for single dilution series including two no template controls. Reactions done in solution. IDT gBlocks™ gene fragment was used as a proxy target template because SARS-CoV-2 control RNA was not commercially available at the time of this experiment. Copy number indicates total copies of target template per reaction.

FIG. 9: Further testing of NEB primer set¹ by real-time LAMP. Amplification curves (with background corrected) shown for duplicate serial dilutions including four no template controls. Reactions done with lyophilized reagents (preliminary formulation before optimization). IDT gBlocks™ gene fragment was used as a proxy target template because SARS-CoV-2 control RNA was not commercially available at the time of this experiment. Copy number indicates total copies of target template per reaction.

FIG. 10: Further testing of Shenyang primer set by real-time LAMP. Amplification curves (with background corrected) shown for duplicate serial dilutions including four no template controls. Reactions done with lyophilized reagents (preliminary formulation before optimization). IDT gBlocks™ gene fragment was used as a proxy target template because SARS-CoV-2 control RNA was not commercially available at the time of this experiment. Copy number indicates total copies of target template per reaction

FIGS. 11A-11C: Initial testing of New York primer set³ by RT real-time LAMP. In each panel, fluorescence curves are shown for a single dilution series including two non-template controls. Target template was SARS-CoV-2 synthetic control RNA from Twist Biosciences. Copy number indicates total copies of target template per reaction. (FIG. 11A) Solution-based RT-LAMP, 1st replicate. (FIG. 11B) Solution-based RT-LAMP, 2nd replicate. (FIG. 11C) RT-LAMP from lyophilized reagents (preliminary formulation before optimization).

FIGS. 12A-12C: Initial testing of Harvard primer set by RT real-time LAMP. In each panel, fluorescence curves are shown for a single dilution series including two non-template controls. Target template was SARS-CoV-2 synthetic control RNA from Twist Biosciences. Copy number indicates total copies of target template per reaction. (FIG. 12A) Solution-based RT-LAMP, 1st replicate. (FIG. 12B) Solution-based RT-LAMP, 2nd replicate. (FIG. 12C) RT-LAMP from lyophilized reagents (preliminary formulation before optimization).

FIGS. 13A-13B: Analytical sensitivity comparison of two lyophilized RT-LAMP formulations. (FIG. 13A) 3M lyo+GuHCl sol. (FIG. 13B) (3M+GuHCl) lyo. All lyo samples were stored at room temperature for 1 day before the RT-LAMP experiment. Fresh samples were prepared on the day of the RT-LAMP experiment and were included in the experiment as reference. Each reaction was incubated at the indicated optimal temperature. Column numbers indicate total RNA copies per 20 μL reaction. False positives are marked by white asterisks.

FIGS. 14A-14B: Further sensitivity analysis of two lyophilized RT-LAMP formulations. (FIG. 14A) 3M lyo+GuHCl sol. (FIG. 14B) (3M+GuHCl) lyo. All lyo samples were stored at room temperature for 1 day before the RT-LAMP experiment. Each reaction was incubated at the indicated optimal temperature. Column numbers indicate total RNA copies per 20 μL reaction. False positives are marked by white asterisks.

FIGS. 15A-15B: Performance of the lyophilized test using the Harvard primer set after 10-day storage in fridge (4° C.). (FIG. 15A) Analytical validation of test sensitivity and specificity for the optimal (3M+GuHCl) lyo formulation. (FIG. 15B) Performance of the test lyophilized with 3M trehalose without GuHCl True negatives and true positives are labeled by (−) and (+), respectively. The importance of temperature optimization is apparent as shown by results in (FIG. 15B).

FIG. 16: Performance of the lyophilized test using the Color Genomics primer set after 10-day storage in the fridge (4° C.). RT-LAMP incubation temperature at 65.5° C. True negatives and true positives are labeled by (−) and (+), respectively. False positives are marked by white asterisks.

FIG. 17: Lyophilization cycle optimization for extended room temperature storage. (A) 1-hr lyophilization. (B) 1-hr lyophilization with 10-minute 45° C. secondary drying. (C) 1-hr lyophilization with 30-minute 45° C. secondary. (D) 1-hr lyophilization with 1-hr 45° C. secondary drying. (E) 1-hr lyophilization without using vacuum concentrator. (A) to (D) were carried out in a vacuum concentrator connected to the lyophilizer. Top panel shows the appearance of the RT-LAMP test kit after lyophilization, storage, and reconstitution. Bottom panel shows RT-LAMP test results after 10-day storage at room temperature. Best performing sets are labeled by green boxes. False positives are marked by white asterisks.

### DETAILED DESCRIPTION

Access to fast and reliable nucleic acid testing continues to play a key role in controlling the SARS-CoV-2 pandemic especially in the context of increased vaccine break-through risks due to new variants. Embodiments of the present disclosure provide a rapid, low-cost (˜2 USD), simple-to-use nucleic acid test kit for self-administered at-home testing without lab instrumentation. The entire sample-to-answer workflow takes <60 minutes, including noninvasive sample collection, one-step RNA preparation, reverse-transcription loop-mediated isothermal amplification (RT-LAMP) in a thermos, and direct visual inspection of colorimetric test result. To facilitate long-term storage without cold chain, a fast one-pot lyophilization protocol was developed to preserve all required biochemical reagents of the colorimetric RT-LAMP test in a single microtube. Notably, the lyophilized RT-LAMP assay demonstrated reduced false positives as well as enhanced tolerance to a wider range of incubation temperatures compared to solution-based RT-LAMP reactions. Validation tests conducted on simulated SARS-CoV-2 infected samples confirmed rapid detection of multiple variants of SARS-CoV-2 virus from both anterior nasal swabs and gingival swabs. With a simple change of primer set, the lyophilized RT-LAMP home test described herein can be easily adapted as a low-cost surveillance platform for other pathogens and infectious diseases of global public health importance.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

“Subject” and “patient” as used herein interchangeably refers to any vertebrate, including, but not limited to, a mammal and a human. In some embodiments, the subject may be a human or a non-human. The subject or patient may be undergoing other forms of treatment.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The terms “administration of” and “administering” a composition as used herein refers to providing a composition of the present disclosure to a subject in need of treatment. The compositions of the present disclosure may be administered by topical (e.g., in contact with skin or surface of body cavity), oral, parenteral (e.g., intramuscular, intraperitoneal, intravenous, ICV, intracisternal injection or infusion, subcutaneous injection, or implant), by spray, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Coefficient of variation” (CV), also known as “relative variability,” is equal to the standard deviation of a distribution divided by its mean.

“Component,” “components,” or “at least one component,” refer generally to a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assessing a test sample, such as a urine, saliva, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Dynamic range” as used herein refers to range over which an assay readout is proportional to the amount of target molecule or analyte in the sample being analyzed. The dynamic range can be the range of linearity of the standard curve.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e. a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Sensitivity” as used herein generally refers to the percentage of true positives that are predicted by a test to be positive, while “specificity,” as used herein refers to the percentage of true negatives that are predicted by a test to be negative. For example, a ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). However, it is well-known that reference levels may vary depending on the nature of the assay used and that assays can be compared and standardized. It further is well within the ordinary skill of one in the art to adapt the disclosure herein for other assays to obtain specific reference levels for those other assays based on the description provided by this disclosure. Whereas the precise value of the reference level may vary between assays, the findings as described herein should be generally applicable and capable of being extrapolated to other assays.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeably and may be a sample of blood, such as whole blood, tissue, skin, urine, serum, plasma, saliva, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

### 2. Detection Assays

Embodiments of the present disclosure provide an inexpensive, one-pot lyophilized colorimetric RT-LAMP molecular test kit for self-administered COVID-19 diagnosis. In addition to its low cost and simplicity, the test kit features a user-friendly home testing workflow that can be easily completed in under 1 hour with no specialized instrumentation or trained personnel (Table 1).

TABLE 1
Features and advantages of the COVID-19 molecular home test kit.

| | |
|---|---|
| Low cost | Total cost per all-in-one kit ~2 USD |
| Simple, noninvasive, | No need for standard/specialized lab equipment or trained personnel. |
| reliable workflow | Self-sample collection with minimal discomfort (anterior nasal swab or gingival swab). |
| | Simple & reproducible testing workflow with minimal liquid transfer steps. |
| | Tolerant of temperature deviations in regular thermoses. |
| Rapid & visual result | Sample-to-result under 1 hour. |
| | Simple visual inspection of colorimetric test readout. |
| Sensitive & accurate | Tolerant to large sample input volume: 20 μL sample directly reconstitutes RT-LAMP. |
| RT-LAMP assay | Excellent analytical limit of detection: 100 RNA copies per reaction (i.e., 5 copies/μL). |
| | Excellent analytical sensitivity: ≥95% |
| | Excellent analytical specificity: >99% |
| | Detects multiple SARS-CoV-2 variants. |
| Long-term stability | Stable for ≥30 days at typical home-refrigeration temperature (4° C.). |
| | Stable for ≥10 days at room temperature (~20 to 22° C.). |
| Easy to manufacture | Fast (2 hr) one-pot lyophilization preserves all test regents in a single microtube. |
| | Extraction-free RNA preparation with inexpensive shelf-stable reagents. |
| Easy to distribute | Compact all-in-one home test kit. |
| | Minimal accessories required (thermos, thermometer, ice).* |
| | Good stability for potential over-the-counter distribution without cold chain. |

*The thermos/thermometer can be replaced by other low-cost solutions depending on use case.

Specifically, embodiments of the present disclosure provide a simple one-pot protocol for lyophilizing colorimetric RT-LAMP. All reagents needed for the isothermal amplification reaction can be quickly preserved in a single microtube, facilitating long-term storage, inexpensive distribution, and simple testing workflow without multiple liquid transfers. Unlike prior work of lyophilized LAMP/RT-LAMP that requires sophisticated lab procedures to separately lyophilize the enzymes from the reaction buffers, the simplicity and robustness of the one-pot lyophilization protocol makes it easy to inexpensively manufacture the molecular test kits at scale. The RT-LAMP assay was tested in regular thermoses and verified its tolerance to temperature deviations in different thermoses. Notably, the test conveniently tolerates larger sample input volumes (i.e., as opposed to 1 μL to 5 μL sample volume commonly used in RT-LAMP assays, the test directly accepts 20 μL swab sample to rehydrate the lyophilized reagents for a 20 μL RT-LAMP reaction). Furthermore, in contrast to conventional molecular diagnostics that usually involve multiple precise volume liquid transfers, the test requires only a single pipetting step (using a low-cost disposable transfer pipette) during the entire testing workflow.

Compared to other COVID-19 testing platforms currently available, the rapid molecular test kit of the present disclosure shows good promises to enable affordable and frequent at-home testing. Due to its low cost and simplicity, the test can allow mass manufacturing in a short timeframe to potentially address the pressing need for global population-scale surveillance, especially in resource-limited regions where COVID-19 is still raging and vaccinations are lagging. For users who cannot conveniently perform the test at home, the test kits can also be readily used at point-of-care settings such as local pharmacies or mobile laboratories, where batch testing of samples can be easily conducted on site using a dedicated dry or water bath or a similar heat source. The patient would still self-collect a sample using the provided swab with the collection tube and then return the sample to the pharmacy. Due to the fast turnaround of the RT-LAMP assay of the present disclosure, the test result can be returned to the patient in under one hour.

Advantages of using the tests described herein for point-of-care/near-patient testing include the further simplified testing workflow and the improved quality control of the tests. However, this alternative configuration may require a technician to handle the patient samples, thus enhanced precautions must be carefully followed (e.g., use of PPE, hand hygiene, frequent instrument decontamination) and assure that all samples are fully inactivated upon receipt. Ideally, the sample handling protocol would be automated at the pharmacy to reduce the risk and improve the testing throughput. Furthermore, it is important to note that the all-in-one lyophilized colorimetric RT-LAMP test kit can be quickly adapted to detect different RNA or DNA targets by simply changing the primer set. This remarkable flexibility coupled with the simplicity and reliability of the test kit and testing workflow hold great promises to enable a robust model platform for low-cost decentralized surveillance of other pathogens (e.g., viruses, bacteria, fungi), including infectious diseases of global public health importance (e.g., dengue, tuberculosis, malaria).

In accordance with the above description, embodiments of the present disclosure include compositions and methods related to the detection and/or quantification of viral RNA in a sample from a subject that has, or is suspected of having, a SARS-CoV-2 infection. Using rapid reverse-transcription loop-mediated isothermal amplification (RT-LAMP), the compositions and methods of the present disclosure provide a portable, inexpensive, rapid, and accurate assay platform for detecting and/or quantifying the presence of a pathogenic organism (e.g., SARS-CoV-2) in a patient sample. In Step 1, a subject self-collects a sample using an anterior nasal swab or gingival swab. In Step 2, the subject plunges the swab into the media inside the collection tube. The subject then gently rubs and/or rolls the swab against the tube wall (e.g., about 10 rolls). The subject then squeezes out the remaining liquid by pressing the swab against the side of the tube, then discards the swab and recaps the tube. In Step 3, the subject adds hot water into a thermos bottle (e.g., 95° C.). Ensuring the sample collection tube lid is tightly secured, the subject places it inside the thermos. The subject then closes the bottle and incubates for 10 about minutes. In Step 4, the subject removes the collection tube and cools it on ice, allowing any debris to settle to the bottom. The subject then uses a disposable transfer pipette to draw about 20 μL of sample from the collection tube. In Step 5, the subject dispenses the sample into the lyophilized RT-LAMP reaction tube. The subject recaps and gently agitates the reaction tube to resuspend the mix (avoiding introducing bubbles). In Step 6, the subject uses a thermometer or a temperature sticker (included in the kit) to adjust the water temperature to ˜67° C. in the thermos bottle. The subject then assembles the RT-LAMP reaction tube into the foam floater and places it in the bottle. The subject closes the bottle and incubates for about 40 minutes. In Step 7, the subject removes the reaction tube and cools it on ice. A visual inspection can then be performed to determine whether the subject's sample is positive or negative for COVID-19 (pink=negative; yellow=positive).

In accordance with the above methods, the present disclosure includes a composition for performing a reverse transcription loop-mediated isothermal amplification (RT-LAMP) reaction. In some embodiments, the composition includes a reaction buffer comprising a DNA polymerase and a reverse transcriptase, a chaotropic agent, and at least one excipient. In some embodiments, the composition is lyophilized to form an RT-LAMP reaction mixture.

In some embodiments, the chaotropic agent includes, but is not limited to n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea. In some embodiments, more than one chaotropic agent can be included in the compositions of the present disclosure. In some embodiments, the chaotropic agent is present at a concentration prior to lyophilization sufficient to produce a reconstituted concentration (i.e., final concentration of the reconstituted composition) ranging from about 5 mM to about 100 mM. In some embodiments, the chaotropic agent is present at a concentration prior to lyophilization sufficient to produce a reconstituted concentration (i.e., final concentration of the reconstituted composition) ranging from about 25 mM to about 100 mM. In some embodiments, the chaotropic agent is present at a concentration prior to lyophilization sufficient to produce a reconstituted concentration (i.e., final concentration of the reconstituted composition) ranging from about 50 mM to about 100 mM. In some embodiments, the chaotropic agent is present at a concentration prior to lyophilization sufficient to produce a reconstituted concentration (i.e., final concentration of the reconstituted composition) ranging from about 75 mM to about 100 mM. In some embodiments, the chaotropic agent is present at a concentration prior to lyophilization sufficient to produce a reconstituted concentration (i.e., final concentration of the reconstituted composition) ranging from about 5 mM to about 75 mM. In some embodiments, the chaotropic agent is present at a concentration prior to lyophilization sufficient to produce a reconstituted concentration (i.e., final concentration of the reconstituted composition) ranging from about 5 mM to about 50 mM. In some embodiments, the chaotropic agent is present at a concentration prior to lyophilization sufficient to produce a reconstituted concentration (i.e., final concentration of the reconstituted composition) ranging from about 5 mM to about 25 mM. In some embodiments, the chaotropic agent is present at a concentration prior to lyophilization sufficient to produce a reconstituted concentration (i.e., final concentration of the reconstituted composition) ranging from about 25 mM to about 75 mM. In some embodiments, the chaotropic agent is present at a concentration prior to lyophilization sufficient to produce a reconstituted concentration (i.e., final concentration of the reconstituted composition) ranging from about 25 mM to about 50 mM.

In some embodiments, the chaotropic agent is guanidine hydrochloride. In some embodiments, the guanidine hydrochloride is present at a concentration prior to lyophilization sufficient to produce a reconstituted concentration (i.e., final concentration of the reconstituted composition) ranging from about 20 mM to about 80 mM. In some embodiments, the guanidine hydrochloride is present at a concentration prior to lyophilization sufficient to produce a reconstituted concentration (i.e., final concentration of the reconstituted composition) ranging from about 40 mM to about 80 mM. In some embodiments, the guanidine hydrochloride is present at a concentration prior to lyophilization sufficient to produce a reconstituted concentration (i.e., final concentration of the reconstituted composition) ranging from about 60 mM to about 80 mM. In some embodiments, the guanidine hydrochloride is present at a concentration prior to lyophilization sufficient to produce a reconstituted concentration (i.e., final concentration of the reconstituted composition) ranging from about 20 mM to about 60 mM. In some embodiments, the guanidine hydrochloride is present at a concentration prior to lyophilization sufficient to produce a reconstituted concentration (i.e., final concentration of the reconstituted composition) ranging from about 20 mM to about 40 mM. In some embodiments, the guanidine hydrochloride is present at a concentration prior to lyophilization sufficient to produce a reconstituted concentration (i.e., final concentration of the reconstituted composition) ranging from about 40 mM to about 80 mM. In some embodiments, the guanidine hydrochloride is present at a concentration prior to lyophilization sufficient to produce a reconstituted concentration (i.e., final concentration of the reconstituted composition) ranging from about 40 mM to about 60 mM.

In some embodiments, the composition for performing an RT-LAMP reaction, as described herein, includes at least one excipient. In some embodiments, the excipient includes, but is not limited to, sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide. In some embodiments, more than one excipient can be included in the compositions of the present disclosure. In some embodiments, the excipient is present at a concentration prior to lyophilization sufficient to produce a reconstituted concentration ranging from about 1% w/v to about 100% w/v. In some embodiments, the excipient is present at a concentration prior to lyophilization sufficient to produce a reconstituted concentration ranging from about 25% w/v to about 100% w/v. In some embodiments, the excipient is present at a concentration prior to lyophilization sufficient to produce a reconstituted concentration ranging from about 50% w/v to about 100% w/v. In some embodiments, the excipient is present at a concentration prior to lyophilization sufficient to produce a reconstituted concentration ranging from about 75% w/v to about 100% w/v. In some embodiments, the excipient is present at a concentration prior to lyophilization sufficient to produce a reconstituted concentration ranging from about 1% w/v to about 75% w/v. In some embodiments, the excipient is present at a concentration prior to lyophilization sufficient to produce a reconstituted concentration ranging from about 1% w/v to about 50% w/v. In some embodiments, the excipient is present at a concentration prior to lyophilization sufficient to produce a reconstituted concentration ranging from about 1% w/v to about 25% w/v. In some embodiments, the excipient is present at a concentration prior to lyophilization sufficient to produce a reconstituted concentration ranging from about 25% w/v to about 75% w/v.

In some embodiments, the excipient is trehalose. In some embodiments, the trehalose is present at a concentration prior to lyophilization sufficient to produce a reconstituted concentration ranging from about 5% w/v to about 20% w/v. In some embodiments, the trehalose is present at a concentration prior to lyophilization sufficient to produce a reconstituted concentration ranging from about 10% w/v to about 20% w/v. In some embodiments, the trehalose is present at a concentration prior to lyophilization sufficient to produce a reconstituted concentration ranging from about 15% w/v to about 20% w/v. In some embodiments, the trehalose is present at a concentration prior to lyophilization sufficient to produce a reconstituted concentration ranging from about 5% w/v to about 15% w/v. In some embodiments, the trehalose is present at a concentration prior to lyophilization sufficient to produce a reconstituted concentration ranging from about 5% w/v to about 10% w/v. In some embodiments, the trehalose is present at a concentration prior to lyophilization sufficient to produce a reconstituted concentration ranging from about 10% w/v to about 15% w/v.

In some embodiments, the composition for performing an RT-LAMP reaction, as described herein, includes a visible pH indicator. In some embodiments, the visible pH indicator includes, but is not limited to, cresol red, phenol red, neutral red, and m-cresol purple. In some embodiments, the composition includes more than one visible pH indicator.

In some embodiments, the composition for performing an RT-LAMP reaction, as described herein, includes one or more primers to carry out the reaction. In some embodiments, the composition comprises a LAMP primer mix comprising F3 primers, B3 primers, FIP primers, BIP primers, LoopF primers, and LoopB primers (e.g., SEQ ID NOs: 1-6, or SEQ ID NOs: 7-12). In some embodiments, the primers comprise the following sequences: F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6. In some embodiments, the primers comprise the following sequences: F3 primer of SEQ ID NO: 7; B3 primer of SEQ ID NO: 8; FIP primer of SEQ ID NO: 9; BIP primer of SEQ ID NO: 10; LoopF primer of SEQ ID NO: 11; and LoopB primer of SEQ ID NO: 12.

In some embodiments, the LAMP primer mix comprises primers specific for cDNA sequences corresponding to RNA from a pathogenic organism. In some embodiments, the pathogenic organism is an RNA virus. In some embodiments, the pathogenic organism is a positive-sense single-stranded RNA virus. In some embodiments, the RNA virus is a coronavirus (e.g., Human coronavirus OC43 (HCoV-OC43), β-CoV; Human coronavirus HKU1 (HCoV-HKU1), β-CoV; Human coronavirus 229E (HCoV-229E), α-CoV; Human coronavirus NL63 (HCoV-NL63), α-CoV; Severe acute respiratory syndrome coronavirus (SARS-CoV), β-CoV; Middle East respiratory syndrome-related coronavirus (MERS-CoV), β-CoV; and Severe acute respiratory syndrome coronavirus 2 (SARS-CoV-2), β-CoV), or a variant or derivative thereof. In some embodiments, the pathogenic organism is SARS-CoV-2, or any variant or derivative thereof. In other embodiments, the pathogenic organism is a negative strand RNA virus (e.g., Influenzavirus, Sendai virus, Human parainfluenza virus 1 (hPIV1), Simian virus (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lympocytic choriomeningitis virus (LCMV), Junin virus, and Lessa fever virus). As would be recognized by one of ordinary skill in the art based on the present disclosure, any suitable RT-LAMP primer set can be designed to target a polynucleotide associated with a pathogenic organism, and subsequently used to detect and/or quantify that pathogenic organism using the compositions and methods described further herein

In some embodiments, the composition for performing an RT-LAMP reaction, as described herein, includes one or more additional reagents for carrying out the reaction. In some embodiments, the one or more additional reagents include, but are not limited to, dNTPs, tris hydrochloride, ammonium sulfate, potassium chloride, magnesium sulfate, betaine, and tween 20.

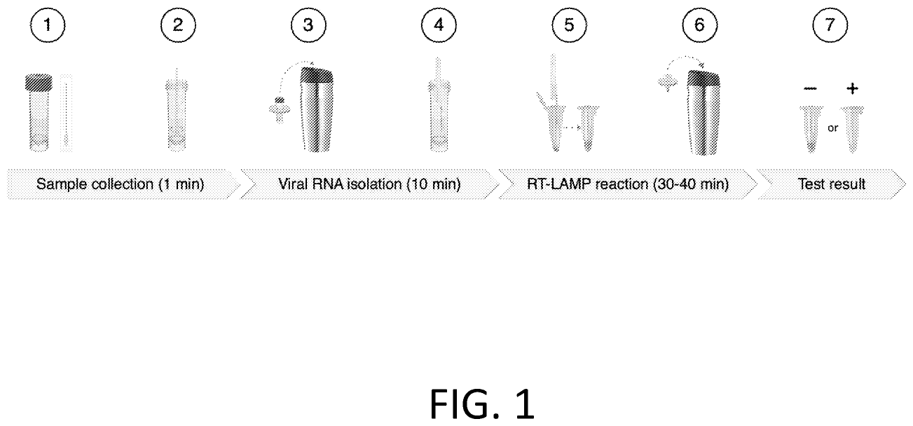

In accordance with the above embodiments, the RT-LAMP reaction mixture as described herein are mixed with a biological sample from a subject or a plurality of subjects (see, e.g., FIG. 1). In some embodiments, the biological sample is obtained or derived from the subject's mouth and/or nasal cavity. As used herein, the terms, “sample,” “test sample,” “specimen,” “sample from a subject,” and “patient sample” may be used interchangeably and may be a sample of blood, such as whole blood, tissue, skin, urine, serum, plasma, saliva, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The sample can be obtained from a subject that has been infected with, or is suspected of being infected with, a pathogenic organism.

Embodiments of the present disclosure also include a method for detecting a pathogenic organism in a biological sample from a subject. In accordance with these embodiments, the method includes: (a) combining in a reaction vessel a biological sample from a subject and the RT-LAMP reaction mixture described herein: (b) incubating the reaction vessel for at least 20 mins at a temperature of at least 60° C.; and (c) performing a visual inspection of the reaction vessel to determine if the biological sample is positive for the presence of a pathogenic organism. In some embodiments, the method further comprises incubating the biological sample for at least 5 mins at a temperature of at least 90° C. prior to step (a).

In some embodiments of the method, the reaction vessel is insulated and configured to contain a liquid at a substantially constant temperature. In some embodiments of the method, the reaction vessel comprises a device for measuring the temperature of the liquid. In some embodiments, the device for measuring the temperature of the liquid can be a thermometer or a temperature sticker.

As described above, the RT-LAMP reaction mixture used according to this method comprises a visible pH indicator selected from the group consisting of cresol red, phenol red, neutral red, and m-cresol purple. In some embodiments of the method, the RT-LAMP reaction mixture further comprises dNTPs, tris hydrochloride, ammonium sulfate, potassium chloride, magnesium sulfate, betaine, and tween 20.

In some embodiments of the method, the RT-LAMP reaction mixture used according to this method comprises a LAMP primer mix that includes one or more primers to carry out the reaction. In some embodiments, the composition comprises a LAMP primer mix comprising F3 primers, B3 primers, FIP primers, BIP primers, LoopF primers, and LoopB primers (e.g., SEQ ID NOs: 1-6, or SEQ ID NOs: 7-12). In some embodiments, the primers comprise the following sequences: F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6. In some embodiments, the primers comprise the following sequences: F3 primer of SEQ ID NO: 7; B3 primer of SEQ ID NO: 8; FIP primer of SEQ ID NO: 9; BIP primer of SEQ ID NO: 10; LoopF primer of SEQ ID NO: 11; and LoopB primer of SEQ ID NO: 12. In some embodiments of the method, the pathogenic organism is an RNA virus. In some embodiments of the method, the pathogenic organism is a positive-sense single-stranded RNA virus. In some embodiments of the method, the RNA virus is a coronavirus (e.g., Human coronavirus OC43 (HCoV-OC43), β-CoV; Human coronavirus HKU1 (HCoV-HKU1), β-CoV; Human coronavirus 229E (HCoV-229E), α-CoV; Human coronavirus NL63 (HCoV-NL63), α-CoV; Severe acute respiratory syndrome coronavirus (SARS-CoV), β-CoV; Middle East respiratory syndrome-related coronavirus (MERS-CoV), β-CoV; and Severe acute respiratory syndrome coronavirus 2 (SARS-CoV-2), β-CoV), or a variant or derivative thereof. In some embodiments of the method, the pathogenic organism is SARS-CoV-2, or a variant thereof. In other embodiments of the method, the pathogenic organism is a negative strand RNA virus (e.g., Influenzavirus, Sendai virus, Human parainfluenza virus 1 (hPIV1), Simian virus (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lympocytic choriomeningitis virus (LCMV), Junin virus, and Lessa fever virus). In some embodiments of the method, the biological sample is obtained from the subject's mouth and/or nasal cavity.

Embodiments of the present disclosure also include a method for generating a reverse transcription loop-mediated isothermal amplification (RT-LAMP) reaction mixture. In accordance with these embodiments, the method includes combining a reaction buffer comprising a DNA polymerase and a reverse transcriptase with a chaotropic agent and at least one excipient into a container; and subjecting the container to a lyophilization process to form an RT-LAMP reaction mixture.

In some embodiments of the method, the chaotropic agent is selected from the group consisting of n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea. In some embodiments of the method, the chaotropic agent is guanidine hydrochloride. In some embodiments of the method, the guanidine hydrochloride is present at a concentration prior to lyophilization sufficient to produce a reconstituted concentration ranging from about 20 mM to about 80 mM.

In some embodiments of the method, the at least one excipient is selected from the group consisting of sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide. In some embodiments of the method, the excipient is trehalose. In some embodiments of the method, the trehalose is present at a concentration prior to lyophilization sufficient to produce a reconstituted concentration ranging from about 5% w/v to about 20% w/v.

In some embodiments of the method, the reaction mixture further comprises a visible pH indicator selected from the group consisting of cresol red, phenol red, neutral red, and m-cresol purple. In some embodiments of the method, the reaction mixture further comprises a LAMP primer mix comprising F3 primers, B3 primers, FIP primers, BIP primers, LoopF primers, and LoopB primers. In some embodiments of the method, the reaction mixture further comprises dNTPs, tris hydrochloride, ammonium sulfate, potassium chloride, magnesium sulfate, betaine, tween 20.

In some embodiments of the method, the LAMP primer mix comprises primers specific for cDNA sequences corresponding to RNA from a pathogenic organism. In some embodiments of the method, the pathogenic organism is an RNA virus. In some embodiments of the method, the pathogenic organism is a positive-sense single-stranded RNA virus. In some embodiments, the RNA virus is a coronavirus (e.g., Human coronavirus OC43 (HCoV-OC43), β-CoV; Human coronavirus HKU1 (HCoV-HKU1), β-CoV; Human coronavirus 229E (HCoV-229E), α-CoV: Human coronavirus NL63 (HCoV-NL63), α-CoV; Severe acute respiratory syndrome coronavirus (SARS-CoV), β-CoV; Middle East respiratory syndrome-related coronavirus (MERS-CoV), β-CoV; and Severe acute respiratory syndrome coronavirus 2 (SARS-CoV-2), β-CoV), or a variant or derivative thereof. In some embodiments of the method, the pathogenic organism is SARS-CoV-2. In other embodiments of the method, the pathogenic organism is a negative strand RNA virus (e.g., Influenzavirus, Sendai virus, Human parainfluenza virus 1 (hPIV1), Simian virus (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lympocytic choriomeningitis virus (LCMV), Junin virus, and Lessa fever virus).

### 3. Materials and Methods

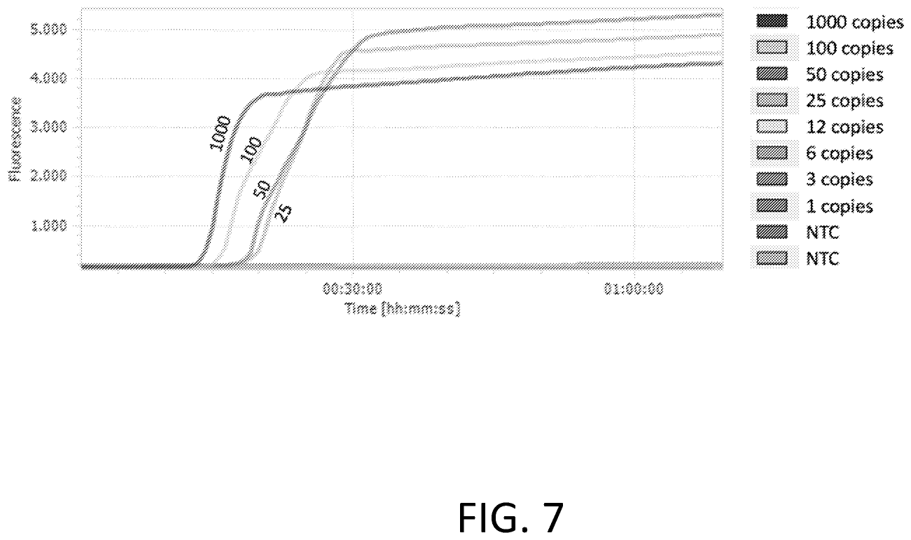

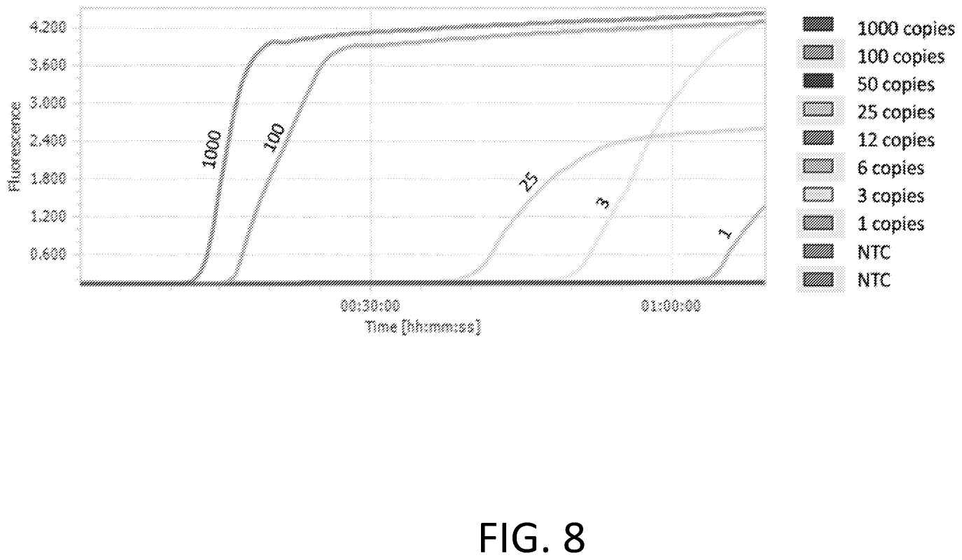

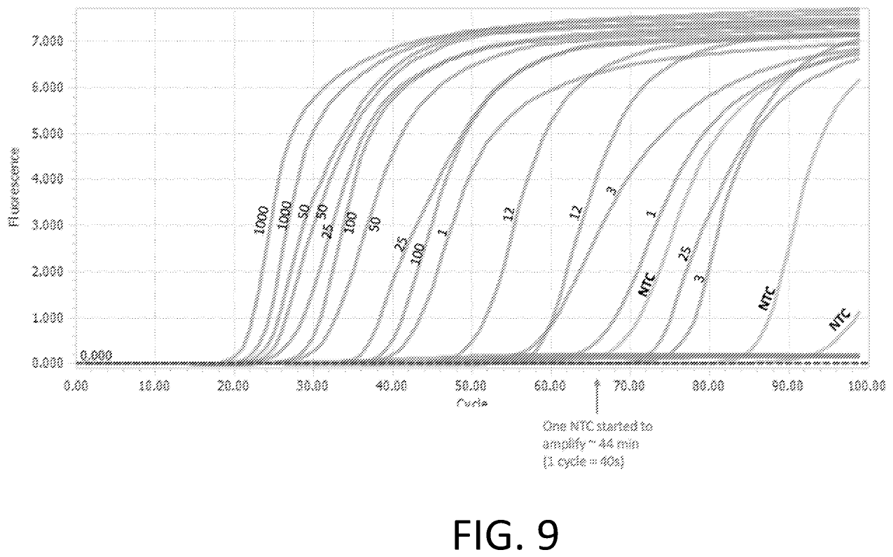

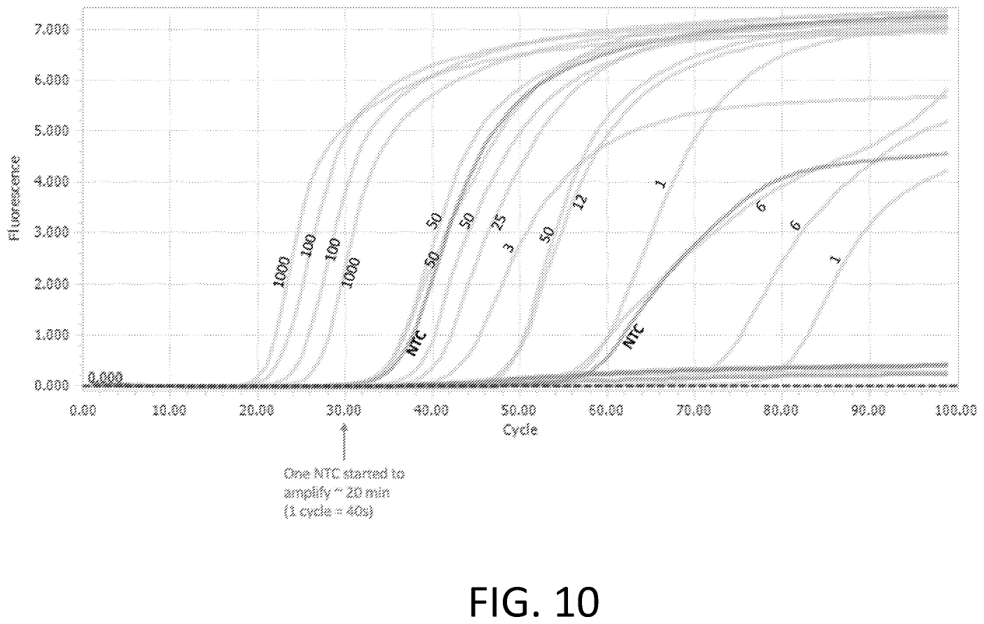

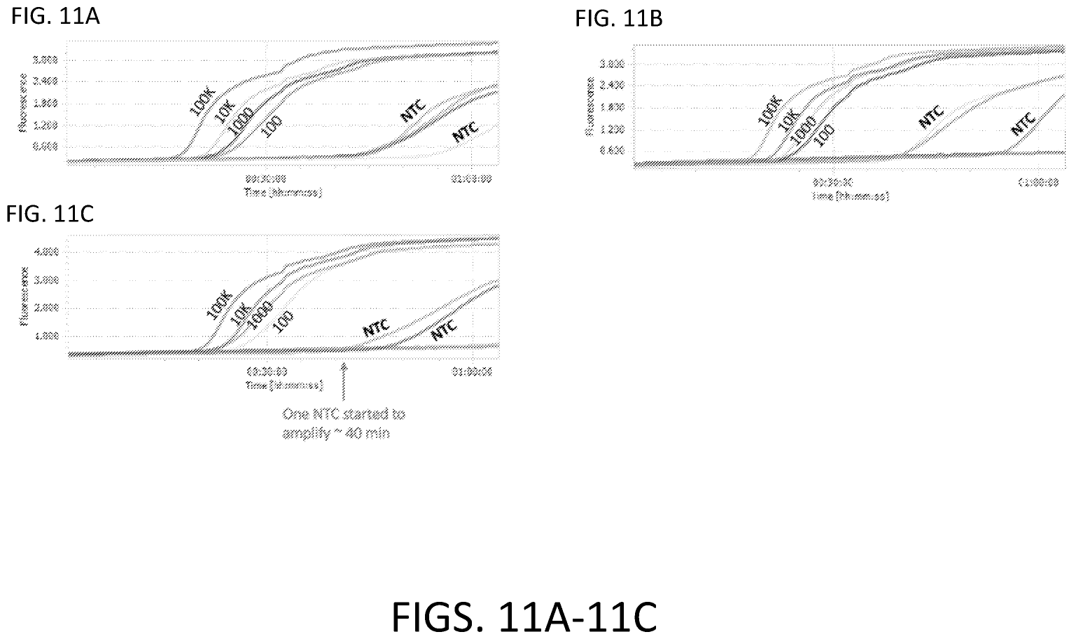

RT-LAMP primers: Several published SARS-CoV-2 RT-LAMP primer sets were carefully screened in terms of the detection sensitivity, false positive and false negative rates, reaction speed, and test reproducibility (FIGS. 7-12). The best performing primer set (Table 2) was selected for further characterization and optimization in the lyophilized colorimetric RT-LAMP home test kit.

TABLE 2
SARS-CoV-2 RT-LAMP primer set used
in the optimized home test kit.

| Primer | Sequence 5′ → 3′ |
|---|---|
| F3 | CGGTGGACAAATTGTCAC |
| | (SEQ ID NO: 1) |
| B3 | CTTCTCTGGATTTAACACACTT |
| | (SEQ ID NO: 2) |
| FIP | TCAGCACACAAAGCCAAAAATT |
| | TATTTTTCTGTGCAAAGGAAAT |
| | TAAGGAG |
| | (SEQ ID NO: 3) |
| BIP | TATTGGTGGAGCTAAACTTAAA |
| | GCCTTTTCTGTACAATCCCTTT |
| | GAGTG (SEQ |
| | ID NO: 4) |
| LoopF | TTACAAGCTTAAAGAATGTCTG |
| | AACACT (SEQ ID NO: 5) |
| LoopB | TTGAATTTAGGTGAAACATTTG |
| | TCACG (SEQ ID NO: 6) |

This primer set targets the ORF1a gene of the SARS-CoV-2 viral genome and is minimally impacted by mutations on current SARS-CoV-2 variants of concern. An additional primer set (Table 3) was tested to confirm the reliable performance of the one-pot lyophilization protocol.

TABLE 3
SARS-CoV-2 RT-LAMP primer set
from the Color Genomics EUA.

| Primer | Sequence 5′ → 3′ |
|---|---|
| F3 | AACACAAGCTTTCGGCAG |
| | (SEQ ID NO: 7) |
| B3 | GAAATTTGGATCTTTGTCATCC |
| | (SEQ ID NO: 8) |
| FIP | TGCGGCCAATGTTTGTAATCAG |
| | CCAAGGAAATTTTGGGGAC |
| | (SEQ ID NO: 9) |
| BIP | CGCATTGGCATGGAAGTCACTT |
| | TGATGGCACCTGTGTAG |
| | (SEQ ID NO: 10) |
| LoopF | TTCCTTGTCTGATTAGTTC |
| | (SEQ ID NO: 11) |
| LoopB | ACCTTCGGGAACGTGGTT |
| | (SEQ ID NO: 12) |

RT-LAMP reagents: WarmStart® Colorimetric LAMP 2X Master Mix (New England Biolabs, cat. M1800L) was used as the RT-LAMP master mix for the test kit. RT-LAMP primers were ordered from IDT as custom DNA oligos with standard desalting. The primers were resuspended in nuclease-free water (Sigma-Aldrich) and mixed to form a 10X primer mix consisting of 2 μM F3 primer, 2 μM B3 primer, 16 μM FIP primer, 16 μM BIP primer, 4 μM LoopF primer, and 4 μM LoopB primer. RT-LAMP reactions were run at 20 μL total reaction volume. Specifically, the lyophilized RT-LAMP reagents were reconstituted with 5 μL sample+15 μL nuclease-free water in all analytical experiments conducted with synthetic SARS-CoV-2 RNA control (Twist Bioscience, cat. 102024). Unless otherwise specified, 20 μL of sample (as opposed to 5 μL sample+15 μL nuclease-free water) was directly added to the lyophilized RT-LAMP mix in validation experiments conducted with contrived SARS-CoV-2 swab samples.

Fast one-pot lyophilization of colorimetric RT-LAMP: The 3M trehalose solution was prepared by dissolving 0.5 g D-(+)-trehalose dihydrate powder (Sigma-Aldrich, M.W. 378.33 g/mol) in 440.5 μL nuclease-free water, followed by vigorous vortexing and heating at 60° C. for 10 minutes to fully dissolve the trehalose to yield a supersaturated solution. This resulted in a solution with a total volume of approximately 760 μL, corresponding to an effective trehalose concentration of around 1.75 M. Because the assay prototyping needed only small amounts of the solutions, to keep the measuring simple and consistent, this resulting solution was referred to as the 3M trehalose throughout the text unless otherwise specified. The trehalose solution was then sterilized by filtering through a 0.2 μm syringe filter (VWR), followed by brief vortex and centrifuge to remove air bubbles. For consistency, the 1M GuHCl solution was prepared similarly by directly dissolving 0.1 g guanidine hydrochloride powder (VWR, M.W. 95.53 g/mol) in 1046.8 μL nuclease-free water without further adjustment of the final volume. The tube containing the GuHCl solution was covered with aluminum foil to protect it from light. Components of the colorimetric RT-LAMP lyophilization formulation were mixed at the specified ratio (Table 4), aliquoted into 0.2 mL PCR tubes, and frozen at −20° C. for 1 hr.

TABLE 4
Optimized formulation of the lyophilized
colorimetric RT-LAMP test.

| Component | Amount used per kit | |
|---|---|---|
| WarmStart ® Colorimetric LAMP 2X Master Mix | 10 | μL |
| 10X Primer Mix | 2 | μL |
| 3M Trehalose | 3 | μL |
| 1M GuHCl | 0.8 | μL |

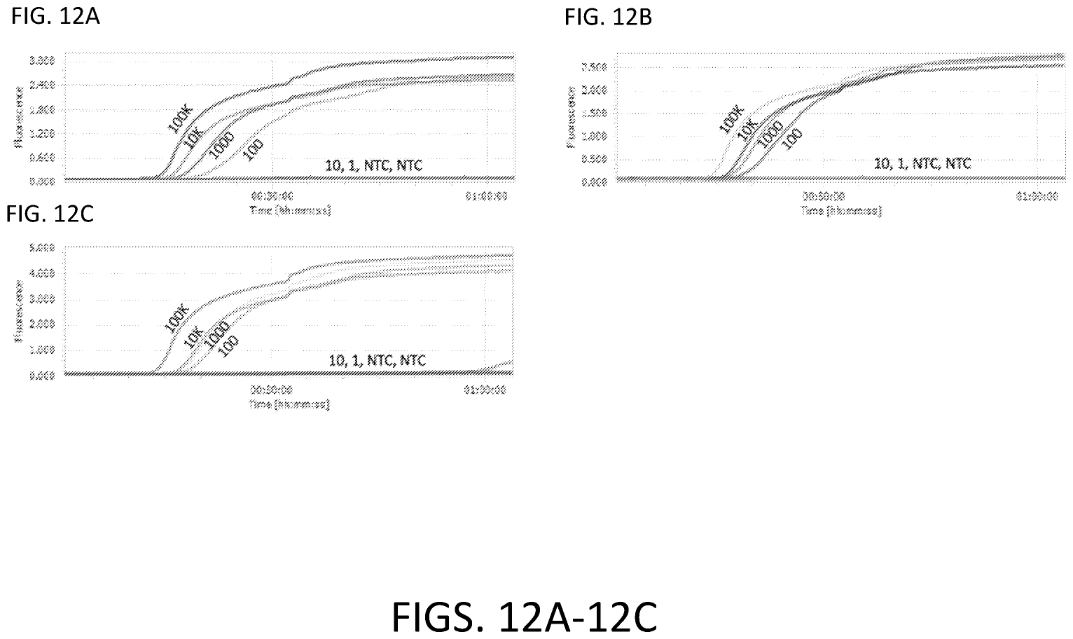

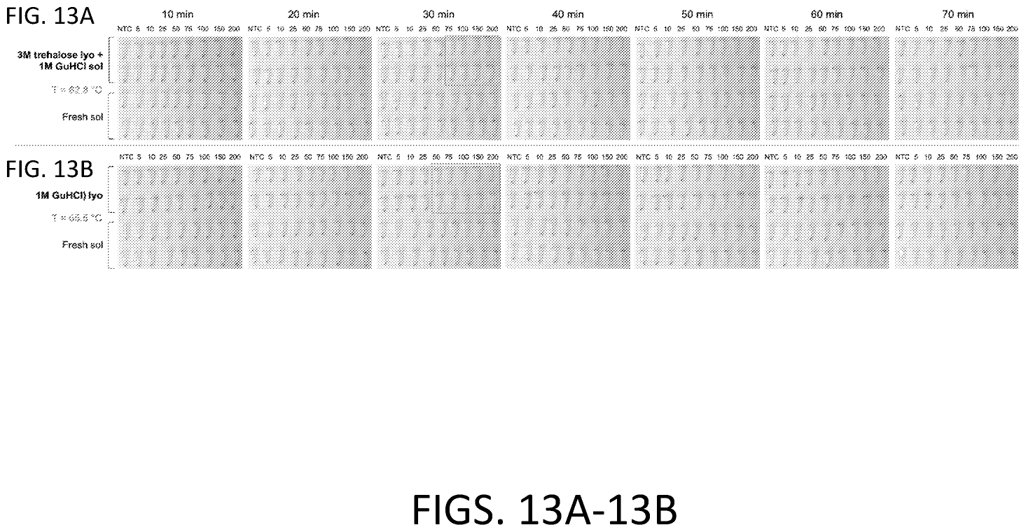

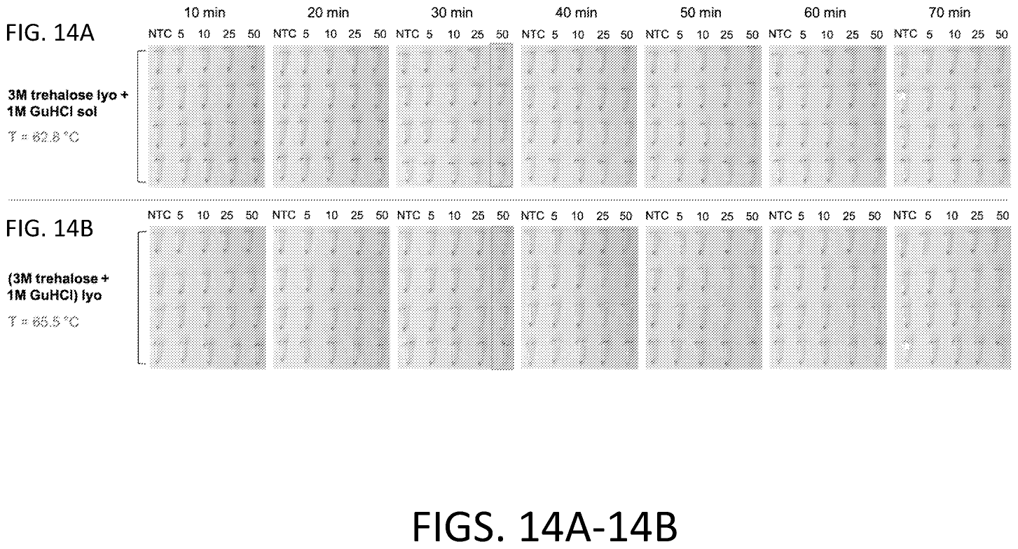

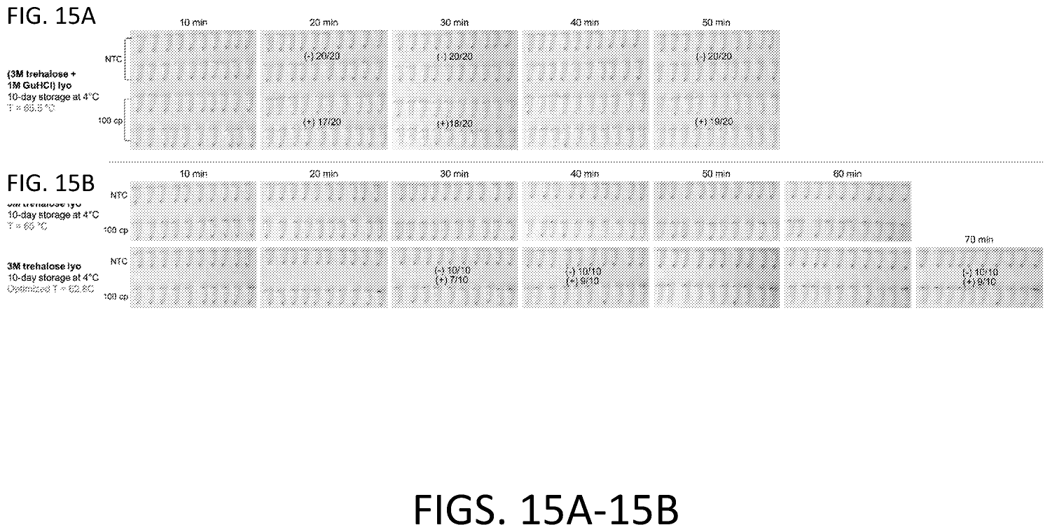

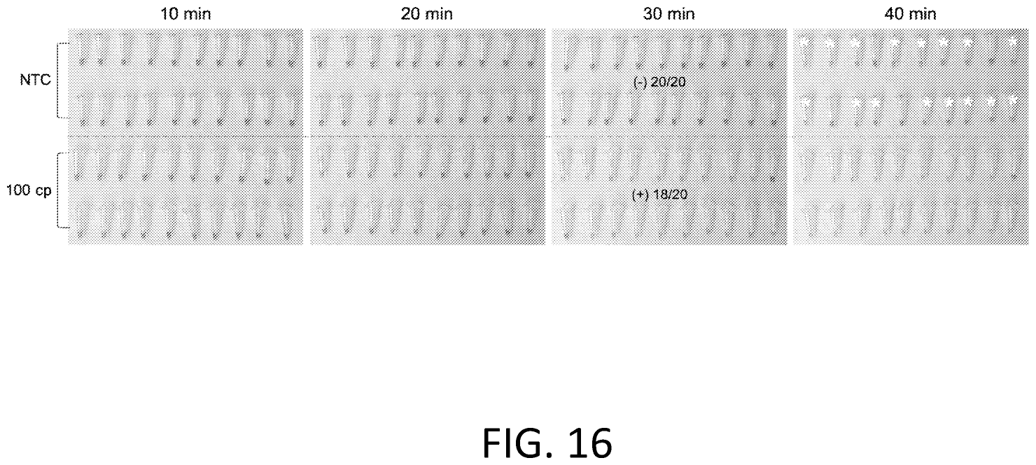

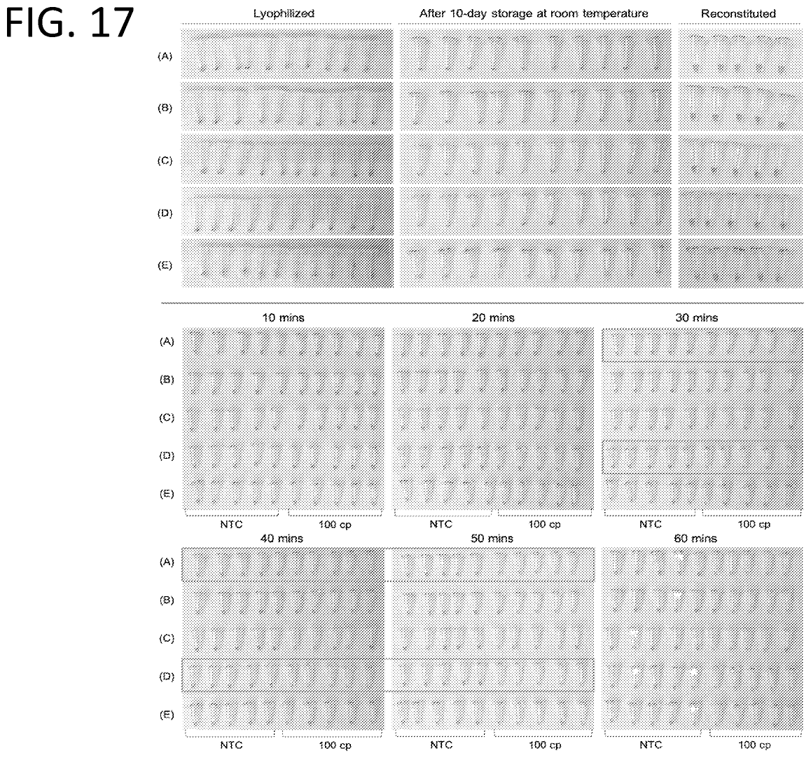

Finally, the tubes were quickly transferred with caps open into a vacuum concentrator (Savant Speedvac SVC-100H) connected to the lyophilizer (VirTis Freezemobile 12SL). Lyophilization was run for 1 hr with the chamber pressure at ˜10 milliTorr and the condenser temperature at ˜−40° C. Details of the RT-LAMP lyophilization protocol optimizations are shown in FIGS. 12-17.

Colorimetric RT-LAMP in thermocycler: RT-LAMP microtubes containing samples or non-template control (NTC) were vortexed, spun down and briefly chilled on ice before pre-incubation photos were taken. RT-LAMP microtubes were incubated in a thermocycler for 60 minutes at the specified temperature, with photos taken at 30, 40, 50 and 60 minutes to assess color change. Tubes were briefly chilled on ice to allow color stabilization, before being photographed. The optimal RT-LAMP incubation temperature was identified by running the reactions with a temperature gradient (T=65° C., G=5° C.) set in a gradient thermal cycler (Eppendorf MasterCycler). To avoid contamination, the RT-LAMP tubes should never be reopened after the incubation reaction.

Colorimetric RT-LAMP in thermos: Both the viral RNA preparation and the RT-LAMP incubation were conducted in thermos. Freshly boiled water was added to pre-warm the thermos for 2 minutes and then dumped out. Next, boiling water was re-added into the thermos and chilled to ˜97° C., after which the virus-spiked samples and NTC (swab media without virus) were incubated for 10 minutes in the thermos (with lid on) and then chilled on ice for 5 minutes to allow cell debris to settle. Next, 20 μL of the heat-inactivated sample “supernatant” (i.e., no cell debris) or NTC were transferred to the RT-LAMP microtube using a disposable transfer pipette. The microtubes were recapped and flicked gently to resuspend the lyophilized RT-LAMP reagents and then chilled on ice before pre-incubation photos were taken. Meanwhile, a mug was filled with boiling water, and allowed to chill to ˜70° C. before pouring into the thermos. The water was allowed to further chill to ˜67° C. before samples were added. Next, the RT-LAMP microtubes were incubated in the thermos with the lid tightly closed for 60 minutes, with photos taken at 30, 40, 50, and 60 minutes. During incubation, the microtubes were secured on a foam floater to ensure that they were vertically and sufficiently submerged in water to activate the RT-LAMP reaction. Finally, the tubes were removed from thermos and briefly chilled on ice to allow color stabilization, before being photographed for test result readout.

TABLE 5
Detailed cost breakdown of the RT-LAMP home test kit.

| Kit component | Item | Vendor | Catalog | Price | Quantity/kit | | Cost/kit |
|---|---|---|---|---|---|---|---|
| Lyophilized RT- | WarmStart ® colorimetric | New | M1800L | $640.64 | 10 | μL | $1.025 |
| LAMP test in | LAMP 2X master mix | England | | | | | |
| single microtube | (DNA & RNA) | BioLabs | | | | | |
| | RT-LAMP primer: F3 | IDT | 100 nmole DNA | $6.30 | 0.004 | nmole | $2.52e−4 |
| | | | Oligo, standard | | | | |
| | | | desalting | | | | |
| | RT-LAMP primer: B3 | IDT | 100 nmole DNA | $7.70 | 0.004 | nmole | $3.08e−4 |
| | | | Oligo, standard | | | | |
| | | | desalting | | | | |
| | RT-LAMP primer: FIP | IDT | 100 nmole DNA | $17.85 | 0.032 | nmole | $5.712e−3 |
| | | | Oligo, standard | | | | |
| | | | desalting | | | | |
| | RT-LAMP primer: BIP | IDT | 100 nmole DNA | $17.15 | 0.032 | nmole | $5.488e−3 |
| | | | Oligo, standard | | | | |
| | | | desalting | | | | |
| | RT-LAMP primer: | IDT | 100 nmole DNA | $9.80 | 0.008 | nmole | $7.84e−4 |
| | LoopF | | Oligo, standard | | | | |
| | | | desalting | | | | |
| | RT-LAMP primer: | IDT | 100 nmole DNA | $9.45 | 0.008 | nmole | $7.56e−4 |
| | LoopB | | Oligo, standard | | | | |
| | | | desalting | | | | |
| | D-(+)-trehalose dihydrate | Sigma- | T9531-25G | $128.00 | 3.405 | mg | $0.017 |
| | | Aldrich | | | | | |
| | Guanidine hydrochloride | VWR | TCG0197-025G | $17.04 | 0.076 | mg | $5.21e−5 |
| | 0.2 mL PCR tube | Sarstedt | 72.737.002 | $19.45 | 1 | | $0.039 |
| Sample collection | Sample collection tube | VWR | 89005-596 | $111.52 | 1 | | $0.112 |
| & viral RNA | Anterior nasal swab | Thomas | MSC-93050D | $35.00 | 1 | | $0.35 |
| isolation | | Scientific | | | | | |
| | Gingival swab | Amazon | B077WQZRTB | $12.99 | 1 | | $0.13 |
| | 20 μL exact volume | Thomas | 783NL | $62.14 | 1 | | $0.124 |
| | transfer pipette | Scientific | | | | | |
| | Nuclease-free water | Sigma- | W4502-1L | $45.76 | 3 | mL | $0.137 |
| | | Aldrich | | | | | |
| | 10X Tris-Borate-EDTA | Sigma- | SRE0062-1L | $72.80 | 0.75 | μL | $5.46e−5 |

| | | | | | | |
|---|---|---|---|---|---|---|
| | (TBE) | Aldrich | | | | |
| Test kit package & | Mylar bag (3 × 4″) | Amazon | B07YM59TDZ | $6.08 | 1 | $0.06 |
| storage | Silica gel packet | Electron | 71206-01 | $15.50 | 1 | $0.16 |
| | | Microscopy | | | | |
| | | Sciences | | | | |
| Accessory* | Temperature sticker | Digi-Sense | UX-09035-52 | $12.37 | 1 | $1.24 |

| Total cost per test kit (for anterior nasal sample): | With accessory: | $3.28 |
|---|---|---|
| | Without accessory: | $2.04 |
| Total cost per test kit (for gingival sample): | With accessory: | $3.06 |
| | Without accessory: | $1.82 |

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Conventional molecular assays do not support convenient use at home by untrained individuals because of the complexity of the testing workflow, dependence on specialized instrumentation, stringent requirement of cold storage for reagents, and the high cost of manufacturing and distribution. To enable truly inexpensive, rapid, reliable at-home testing of COVID-19, a simple all-in-one molecular home test kit was developed based on lyophilized colorimetric RT-LAMP, requiring only a regular thermos and a thermometer to conduct the self-administered test. A rapid, one-pot lyophilization protocol was developed to quickly preserve all reagents needed for the colorimetric RT-LAMP test in a single microtube, facilitating long-term stability, inexpensive distribution, and convenient use of the home test kit. Notably, the lyophilized RT-LAMP assay demonstrated reduced false positives and higher tolerance to a wider range of incubation temperatures compared to conventional solution-based RT-LAMP reactions. To enable detection of viruses from clinical sample matrices, a one-step RNA preparation protocol was adapted based on low-cost shelf-stable reagents. The entire sample-to-answer workflow (FIG. 1) takes <60 minutes, including noninvasive sample collection (anterior nasal swab or gingival swab), quick extraction-free RNA preparation, optimized RT-LAMP reaction in a thermos, and finally a colorimetric interpretation of the test result.

RT-LAMP reactions rely on active enzymatic components (i.e., DNA polymerase and reverse transcriptase) that must be stored at a low temperature (typically −20° C.). To preserve the RT-LAMP reagents for home test use, lyophilization, also known as freeze drying, was employed to extend the shelf-life of the test kit and facilitate simple test kit distribution, handling, and storage under convenient temperatures (e.g., at typical home-refrigeration temperature or at room temperature). A lyophilized test kit also reduces the number of pipetting steps to improve usability and minimize contamination. However, lyophilization is typically an expensive and time-consuming process involving three stages including freezing, primary drying, and secondary drying which can be difficult to design and optimize.

As described further herein, embodiments of the present disclosure include a fast, one-pot lyophilization process that minimizes the drying time by completing both the primary and secondary drying under a single condition. Unlike prior lyophilization protocols developed for molecular biology assays, the protocol of the present disclosure eliminates the need to separately lyophilize the reaction buffer and the enzymes. Instead, the simplified protocol enables one-pot lyophilization of all reagents needed for the colorimetric RT-LAMP in a single microtube (Table 4), and the entire lyophilization process can be completed in under 2 hours. Trehalose and dextran were tested as candidate excipients to provide cryo- and lyoprotection during lyophilization, as well as enhanced stability for long-term storage. In addition, guanidine hydrochloride (GuHCl) was included in the optimized formulation to improve the reaction speed and the sensitivity of colorimetric RT-LAMP. Multiple sets of recently published RT-LAMP primers were tested, and the RT-LAMP assay was optimized with a well-performing primer set (Table 2) which targets the ORF1a gene of the viral genome and is minimally impacted by the mutations from recent SARS-CoV-2 variants.

### Example 2

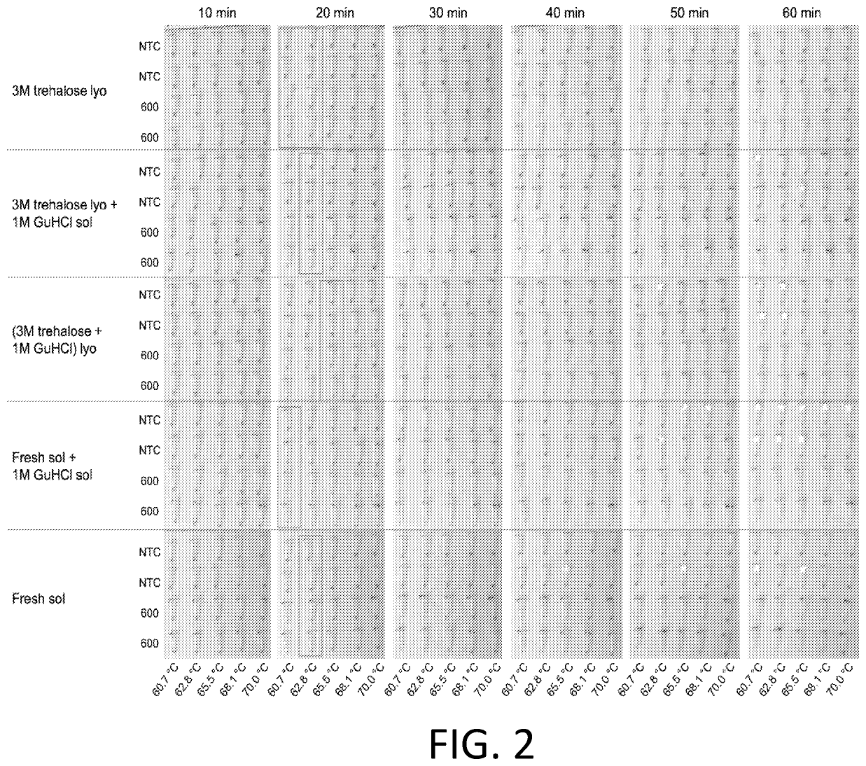

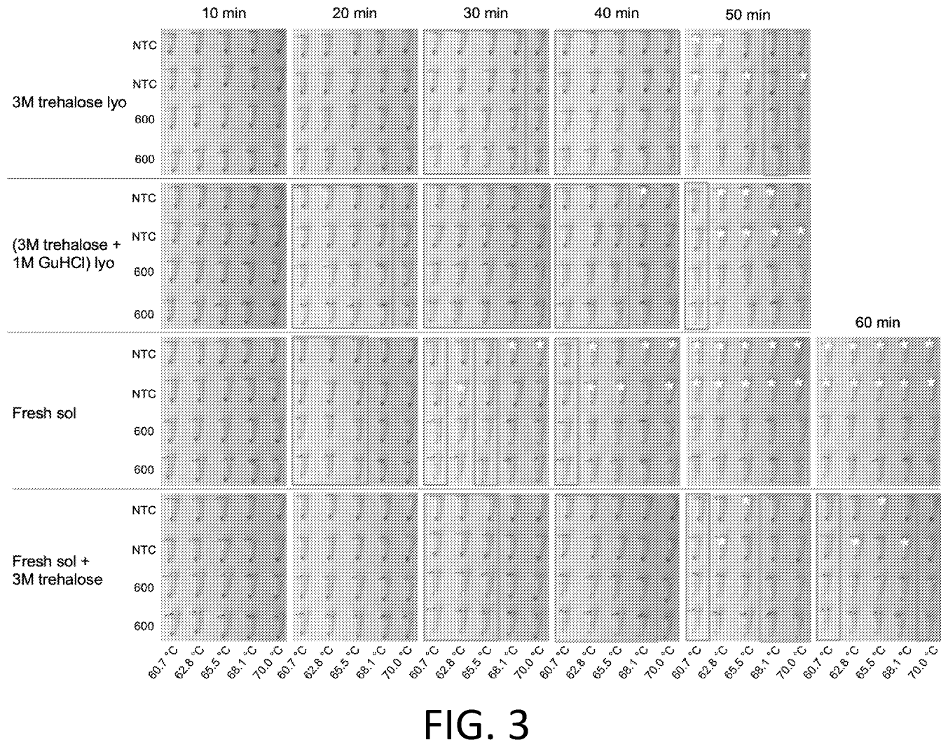

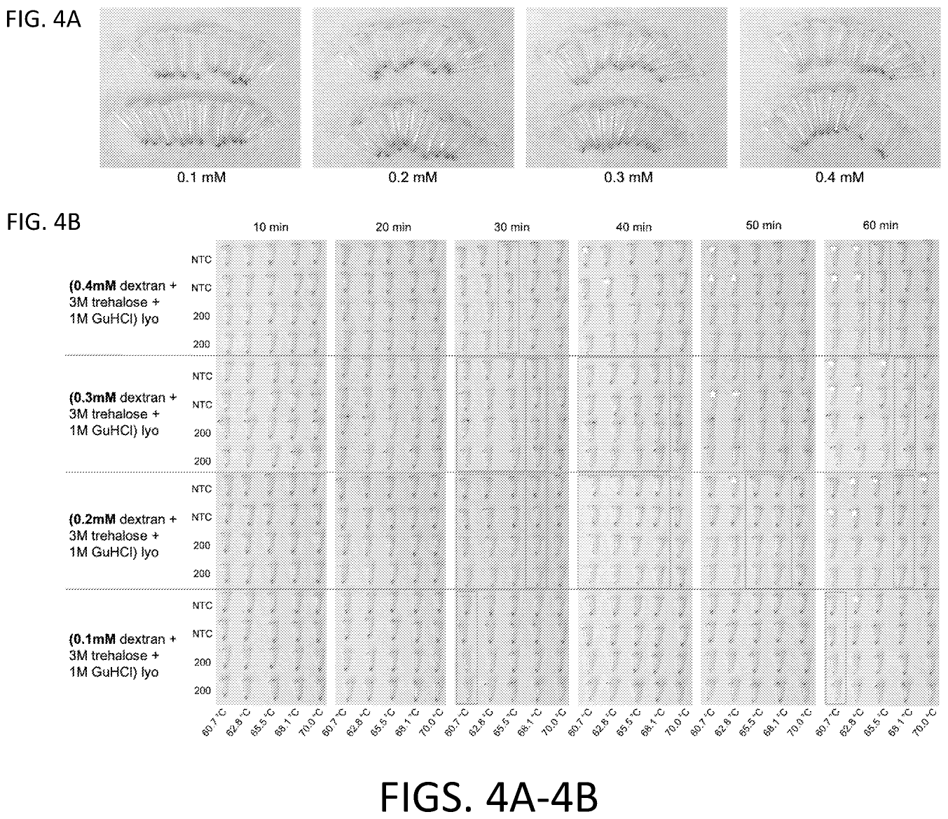

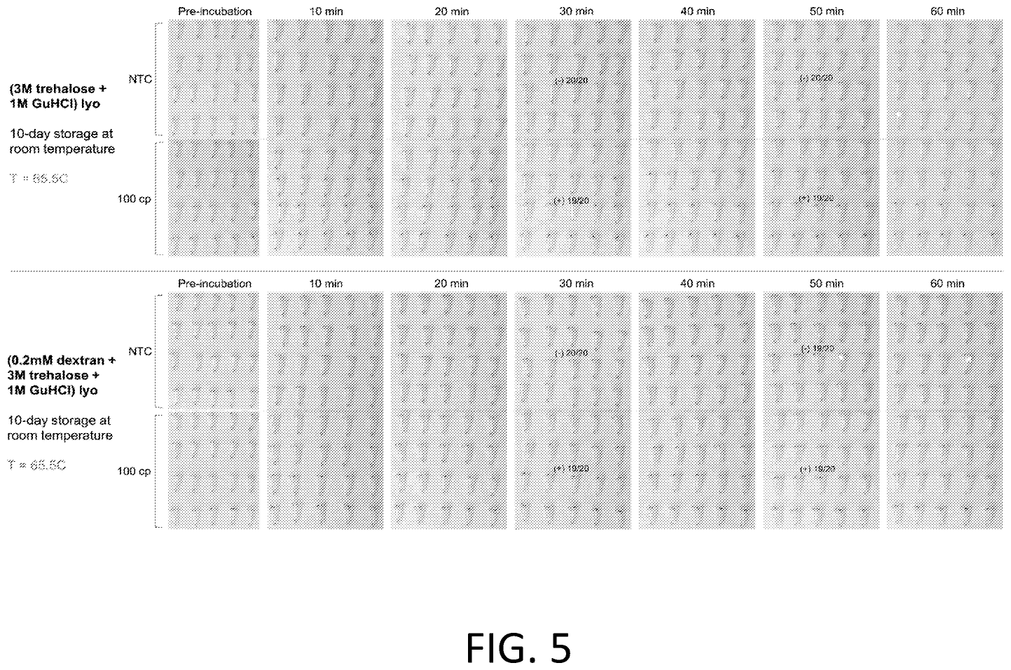

Compared to typical solution-based RT-LAMP reactions, it was observed that the addition of trehalose at the optimized concentration significantly reduced the occurrence of RT-LAMP false positives. The slight decrease in reaction speed was mitigated by the addition of GuHCl. Notably, the lyophilized RT-LAMP reactions also enabled a wider compatible range of incubation temperatures compared to the solution-based RT-LAMP reactions, thus improving the assay's robustness to tolerate the use of regular thermoses for reaction incubation without precise temperature control. Specifically, as shown in FIG. 2, the lyophilized assay (“3M trehalose+1M GuHCl) lyo”) performed robustly across the entire temperature gradient tested (60.7° C.-70.0° C.) with clear readout of true positives as early as 20 minutes and no false positives by 50 minutes of incubation at most temperatures within the temperature gradient. In contrast, the solution-based RT-LAMP assay (“Fresh sol”) based on the same primers and master mix formulation showed a narrower range of compatible temperatures, slower turnaround, and earlier occurrence of false positives. The beneficial effect of the one-pot lyophilization was also observed in FIG. 3, where a similar temperature gradient experiment was conducted for the assay based on a different published RT-LAMP primer set. It was hypothesized that the enhanced performance of the one-pot lyophilized assay is partly due to the inclusion of trehalose in the RT-LAMP formulation. In addition to its role as a lyo-and thermal-protectant, trehalose was found to have a DNA duplex destabilizing effect in prior literature. Such an effect helps to improve the specificity and yield of isothermal amplification reactions, which agrees with the observations from experimental data. The effect of adding dextran (FIG. 4) to potentially extend the shelf-life of the lyophilized RT-LAMP assay at room temperature was also investigated; however, the addition of dextran did not appear to significantly enhance the performance of the lyophilized RT-LAMP assay (FIG. 5).

### Example 3

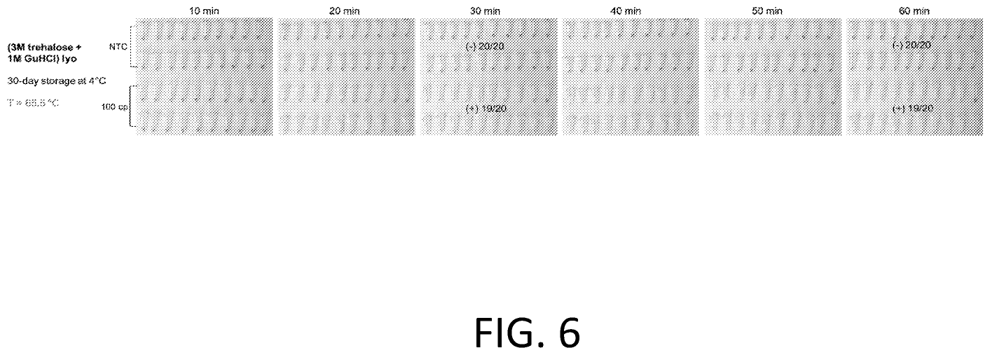

According to in-house validation using synthesized SARS-CoV-2 RNA (Twist Bioscience), the test kit described herein remains stable for at least 10 days at room temperature (˜20 to 22° C.) and 30 days at typical home-refrigeration temperature (4° C.), achieving ≥95% analytical sensitivity and >99% analytical specificity with a reproducible limit of detection down to 100 copies of viral RNA per reaction (i.e., 5 copies/μL) under both storage conditions FIGS. 5 and 6). According to analytical results from contrived anterior nasal swab and gingival swab samples, the test successfully detected multiple SARS-CoV-2 variants and their isolates from different geographical locations. The simplicity of the assay of the present disclosure allows quick change of the primer set to detect emerging variants of SARS-CoV-2 and other pathogens and diseases of public health importance.

What is claimed is:

1. A composition for performing a reverse transcription loop-mediated isothermal amplification (RT-LAMP) reaction, the composition comprising:
a reaction buffer comprising a DNA polymerase and a reverse transcriptase;
a chaotropic agent; and
at least one excipient;
wherein the composition is lyophilized to form an RT-LAMP reaction mixture.

2. The composition of claim 1, wherein the chaotropic agent is selected from the group consisting of n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea.

3. The composition of claim 2, wherein the chaotropic agent is guanidine hydrochloride.

4. The composition of claim 3, wherein the guanidine hydrochloride is present at a concentration prior to lyophilization sufficient to produce a reconstituted concentration ranging from about 20 mM to about 80 mM.

5. The composition of any of claims 1 to 4, wherein the at least one excipient is selected from the group consisting of sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide.

6. The composition of claim 5, wherein the excipient is trehalose.

7. The composition of claim 6, wherein the trehalose is present at a concentration prior to lyophilization sufficient to produce a reconstituted concentration ranging from about 5% w/v to about 20% w/v.

8. The composition of any of claims 1 to 7, wherein the composition further comprises a visible pH indicator selected from the group consisting of cresol red, phenol red, neutral red, and m-cresol purple.

9. The composition of any of claims 1 to 8, wherein the composition further comprises a LAMP primer mix comprising F3 primers, B3 primers, FIP primers, BIP primers, LoopF primers, and LoopB primers.

10. The composition of claim 9, wherein the composition further comprises dNTPs, tris hydrochloride, ammonium sulfate, potassium chloride, magnesium sulfate, betaine, and tween 20.

11. The composition of any of claims 1 to 10, wherein the RT-LAMP reaction mixture is mixed with a biological sample from a subject.

12. The composition of claim 11, wherein the biological sample is obtained from the subject's mouth and/or nasal cavity.

13. The composition of claim 9, wherein the LAMP primer mix comprises primers specific for cDNA sequences corresponding to RNA from a pathogenic organism.

14. The composition of claim 13, wherein the pathogenic organism is an RNA virus.

15. The composition of claim 13 or claim 14, wherein the pathogenic organism is SARS-CoV-2.

16. The composition of claim 15, wherein the primers comprise the following sequences:
F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6.

17. A method for detecting a pathogenic organism in a biological sample from a subject, the method comprising:
(a) combining in a reaction vessel a biological sample from a subject and the RT-LAMP reaction mixture of claim 1;
(b) incubating the reaction vessel for at least 20 mins at a temperature of at least 60° C.; and
(c) performing a visual inspection of the reaction vessel to determine if the biological sample is positive for the presence of a pathogenic organism.

18. The method of claim 17, wherein the method further comprises incubating the biological sample for at least 5 mins at a temperature of at least 90° C. prior to step (a).

19. The method of claim 17 or claim 18, wherein the reaction vessel is insulated and configured to contain a liquid at a substantially constant temperature.

20. The method of claim 19, wherein the reaction vessel comprises a device for measuring the temperature of the liquid.

21. The method of any of claims 17-20, wherein the RT-LAMP reaction mixture further comprises a visible pH indicator selected from the group consisting of cresol red, phenol red, neutral red, and m-cresol purple.

22. The method of any of claims 17-21, wherein the RT-LAMP reaction mixture further comprises dNTPs, tris hydrochloride, ammonium sulfate, potassium chloride, magnesium sulfate, betaine, and tween 20.

23. The method of any of claims 17-22, wherein the RT-LAMP reaction mixture further comprises a LAMP primer mix comprising F3 primers, B3 primers, FIP primers, BIP primers, LoopF primers, and LoopB primers.

24. The method of any of claims 17-23, wherein the LAMP primer mix comprises primers specific for cDNA sequences corresponding to RNA from a pathogenic organism.

25. The method of claim 24, wherein the pathogenic organism is an RNA virus.

26. The method of claim 24 or claim 25, wherein the pathogenic organism is SARS-CoV-2.

27. The method of any of claims 17-26, wherein the biological sample is obtained from the subject's mouth and/or nasal cavity.

28. A method for generating a reverse transcription loop-mediated isothermal amplification (RT-LAMP) reaction mixture, the method comprising:
combining a reaction buffer comprising a DNA polymerase and a reverse transcriptase with a chaotropic agent and at least one excipient into a container; and
subjecting the container to a lyophilization process to form an RT-LAMP reaction mixture.

29. The method of claim 29, wherein the chaotropic agent is selected from the group consisting of n-butanol, ethanol, guanidine hydrochloride, guanidine thiocyanate, lithium perchlorate, lithium acetate, magnesium chloride, phenol, 2-propanol, sodium dodecyl sulfate, sodium iodide, thiourea, and urea.

30. The method of claim 28 or claim 29, wherein the chaotropic agent is guanidine hydrochloride.

31. The method of any of claims 29 to 31, wherein the guanidine hydrochloride is present at a concentration prior to lyophilization sufficient to produce a reconstituted concentration ranging from about 20 mM to about 80 mM.

32. The method of any of claims 28 to 31, wherein the at least one excipient is selected from the group consisting of sucrose, trehalose, dextran, lactose, glucose, raffinose, mannitol, sorbitol, glycine, histidine, arginine, gelatin, dextrose, hydroxyethyl starch, ethylene glycol, propylene glycol, ethylenediamine tetraacetic acid, and dimethyl sulfoxide.

33. The method of claim 32, wherein the excipient is trehalose.

34. The method of claim 33, wherein the trehalose is present at a concentration prior to lyophilization sufficient to produce a reconstituted concentration ranging from about 5% w/v to about 20% w/v.

35. The method of any of claims 28 to 34, wherein the reaction mixture further comprises a visible pH indicator selected from the group consisting of cresol red, phenol red, neutral red, and m-cresol purple.

36. The method of any of claims 28 to 35, wherein the reaction mixture further comprises a LAMP primer mix comprising F3 primers, B3 primers, FIP primers, BIP primers, LoopF primers, and LoopB primers.

37. The method of any of claims 28 to 36, wherein the reaction mixture further comprises dNTPs, tris hydrochloride, ammonium sulfate, potassium chloride, magnesium sulfate, betaine, tween 20.

38. The method of claim 36, wherein the LAMP primer mix comprises primers specific for cDNA sequences corresponding to RNA from a pathogenic organism.

39. The method of claim 38, wherein the pathogenic organism is an RNA virus.

40. The method of claim 37 or 38, wherein the pathogenic organism is SARS-CoV-2.

41. The method of claim 40, wherein the primers comprise the following sequences:
F3 primer of SEQ ID NO: 1; B3 primer of SEQ ID NO: 2; FIP primer of SEQ ID NO: 3; BIP primer of SEQ ID NO: 4; LoopF primer of SEQ ID NO: 5; and LoopB primer of SEQ ID NO: 6.
